(19) United States
(12) Patent Application Publication
(10) Pub. No.: US 2023/0255994 A1
(43) Pub. Date: Aug. 17, 2023

(54) TREATMENT OF COVID-19 AND/OR ACUTE KIDNEY INJURY

(71) Applicant: Metro International Biotech, LLC, Worcester, MA (US)

(72) Inventor: David J. Livingston, Worcester, MA (US)

(21) Appl. No.: 18/013,455

(22) PCT Filed: Jul. 1, 2021
(86) PCT No.: PCT/US21/40102

§ 371 Date: Dec. 28, 2022

Related U.S. Application Data
(60) Provisional application No. 63/046,889, filed on Jul. 1, 2020.

(51) Int. Cl. A61K 31/706

(52) U.S. Cl. CPC A61K 31/706

(57) ABSTRACT

The present disclosure relates to methods of treating disease, such as infectious disease, with NAD boosters. Exemplary NAD boosters include NMN, NR, analogs, prodrugs, salts, and crystals thereof. In some cases, the disease is a viral infection such as coronavirus, SARS, or COVID-19. In some cases, the treatment is for a specific symptom or injury caused by viral infection, preferably for acute kidney injury (AKI). The present disclosure also relates to treatment of acute kidney injury as a result of any cause.

### CROSS-REFERENCE TO RELATED APPLICATIONS

This application claims priority to U.S. Provisional Application No. 63/046,889, filed Jul. 1, 2020, which is incorporated herein by reference in its entirety for all purposes.

### BACKGROUND

Nicotinamide adenine dinucleotide (NAD) and its derivative compounds are known as essential coenzymes in cellular redox reactions in all living organisms. Several lines of evidence have also shown that NAD participates in a number of important signaling pathways in mammalian cells, including poly(ADP-ribosyl)ation in DNA repair, mono-ADP-ribosylation in the immune response and G protein-coupled signaling, and the synthesis of cyclic ADP-ribose and nicotinate adenine dinucleotide phosphate (NAADP) in intracellular calcium signaling. It has also been shown that NAD and its derivatives play an important role in transcriptional regulation. In particular, the discovery of Sir2 NAD-dependent deacetylase activity drew attention to this new role of NAD. However, NAD is an intracellular metabolite, and does not readily lend itself to external supplementation. It has been suggested that utilizing precursors to the natural synthesis of NAD may be an effective way to increase NAD.

Two exemplary precursors that could be administered to increase NAD are nicotinamide mononucleotide (NMN), which is directly synthesized into NAD, and nicotinamide riboside (NR), which is recycled from the utilization of NAD, into NMN. Given the possible therapeutic benefits associated with nicotinic acid mononucleotide and its derivatives, there is a need for improved compositions and methods for using such compositions.

### SUMMARY

NAD boosters such as NMN, NR, analogs thereof, prodrugs thereof, and salts thereof, can improve outcomes for subjects with infections, including viral infections such as SARS-CoV-2 and other coronavirus infections. For example, NAD boosters can help modulate cytokine release, thereby alleviating or preventing cytokine release syndrome (cytokine storm) which is experienced by some patients with COVID-19 or related diseases.

An aspect of the present disclosure provides a method of treating COVID-19 in a human patient comprising administering nicotinamide mononucleotide (NMN) to said patient, wherein said NMN is administered to said patient according to one or more of the following conditions: (i) in the absence of administration of a dose of zinc sulfate, betaine, or a mixture thereof, equal to or greater than the minimum effective dose or minimum recommended dose for any indication in which said zinc sulfate, betaine, or a mixture thereof has regulatory approval or institutional review board approval for administration; (ii) in a patient that has not received azithromycin as treatment for COVID-19 prior to initial administration of NMN; or (iii) in a patient that has not received hydroxychloroquine as treatment for COVID-19 prior to initial administration of NMN.

An aspect of the present disclosure provides a method of treating COVID-19 in a human patient comprising administering nicotinamide mononucleotide (NMN) to said patient, wherein said NMN is administered to said patient in the absence of administration of a dose of zinc sulfate, betaine, or a mixture thereof, equal to or greater than the minimum effective dose or minimum recommended dose for any indication in which said zinc sulfate, betaine, or a mixture thereof has regulatory approval or institutional review board approval for administration.

An aspect of the present disclosure provides a method of treating COVID-19 in a human patient comprising administering nicotinamide mononucleotide (NMN) to said patient contemporaneously with less than the minimum effective dose of zinc sulfate, betaine, or a mixture thereof. In some embodiments, the minimum effective dose of zinc sulfate is about 50 mcg/kg, about 40 mcg/kg, or about 30 mcg/kg. In some embodiments, the amount of zinc sulfate administered is less than about 50 mcg/kg per day, less than about 40 mcg/kg, or less than about 30 mcg/kg. In some embodiments, the amount of zinc sulfate administered is less than about 3.5 mg per day, less than about 3 mg per day, or less than about 2 mg per day. In some embodiments, the minimum effective dose of betaine is about 75 mg/kg per day, about 90 mg/kg per day, or about 100 mg/kg per day. In some embodiments, the amount of betaine administered is less than about 75 mg/kg per day, less than about 90 mg/kg per day, or less than about 100 mg/kg per day betaine. In some embodiments, the amount of betaine administered is less than about 5 grams per day, less than about 6 grams per day, or less than about 7 grams per day betaine. In some embodiments, an amount of zinc sulfate greater than zero but less than the minimum effective dose is administered. In some embodiments, no zinc sulfate is administered. In some embodiments, an amount of betaine greater than zero but less than the minimum effective dose is administered. In some embodiments, no betaine is administered.

An aspect of the present disclosure provides a method of treating COVID-19 in a human patient comprising administering nicotinamide mononucleotide (NMN) to said patient, wherein said patient that has not received azithromycin as a treatment for COVID-19 prior to initial administration of NMN.

An aspect of the present disclosure provides a method of treating COVID-19 in a human patient comprising administering nicotinamide mononucleotide (NMN) to said patient, wherein said patient has not received hydroxychloroquine as a treatment for COVID-19 prior to initial administration of NMN.

In some embodiments, said NMN is administered in crystal form. In some embodiments said NMN is administered in amorphous form. In some embodiments said NMN is administered in a solid pharmaceutical dosage form. In some embodiments said NMN is administered as a tablet. In some embodiments said NMN is administered to reduce or alleviate the symptoms of COVID-19-related cytokine storm in a patient exhibiting symptoms of cytokine storm. In some embodiments said NMN is administered to reduce or prevent the incidence of COVID-19-related cytokine storm in a patient diagnosed with COVID-19 but not yet exhibiting symptoms of cytokine storm. In some embodiments said NMN is administered to reduce or prevent the incidence of, or to reduce or alleviate the symptoms of, COVID-19-related cytokine storm in a patient at risk of developing COVID-19-related cytokine storm. In some embodiments said risk of developing COVID-19-related cytokine storm is evaluated based on clinical presentation of risk factors, or based on a laboratory-confirmed biomarker associated with a risk of developing COVID-19-related cytokine storm. In some embodiments, said NMN is administered to a patient exhibiting AKI (acute kidney injury). In some embodiments, said AKI is evidenced by increased creatinine lab results. In some embodiments said patient is admitted in a hospital or in-patient clinic. In some embodiments said patient is not receiving assistance in breathing from a ventilator. In some embodiments said NMN is administered in a dose of between about 100 mg and about 4 grams per day. In some embodiments said NMN is administered in a dose between about 200 mg and about 2 grams per day. In some embodiments said NMN is administered in a dose between about 400 mg and about 1 gram per day. In some embodiments said NMN is administered in a dose between about 500 mg and about 2 grams per day. In some embodiments, said NMN is administered in a daily dosage regimen selected from once daily to four times daily. In some embodiments said NMN is administered in a daily dosage regimen selected from once daily, twice daily, and four times daily. In some embodiments said NMN is administered for a period up to 5 days inclusive. In some embodiments said NMN is administered for a period greater than 5 days.

An aspect of the present disclosure provides a method of prophylactic treatment of COVID-19 in a human at risk of contracting COVID-19 or in a human with asymptomatic or mild COVID-19, wherein said human has not received a diagnosis of COVID-19 based on a laboratory-confirmed finding of the presence of SARS-CoV-2 virus, said method comprising administering nicotinamide mononucleotide (NMN) to said patient in a daily dose between about 100 mg and 4 grams.

An aspect of the present disclosure provides a method of treating COVID-19 in a human patient comprising administering an NAD booster to said patient, wherein said NAD booster is administered to said patient according to one or more of the following conditions: (i) in the absence of administration of a dose of zinc sulfate, betaine, or a mixture thereof, equal to or greater than the minimum effective dose or minimum recommended dose for any indication in which said zinc sulfate, betaine, or a mixture thereof has regulatory approval or institutional review board approval for administration; (ii) in a patient that has not received azithromycin as treatment for COVID-19 prior to initial administration of said NAD booster; or (iii) in a patient that has not received hydroxychloroquine as treatment for COVID-19 prior to initial administration of said NAD booster.

An aspect of the present disclosure provides a method of treating COVID-19 in a human patient comprising administering an NAD booster to said patient, wherein said NAD booster is administered to said patient in the absence of administration of a dose of zinc sulfate, betaine, or a mixture thereof, equal to or greater than the minimum effective dose or minimum recommended dose for any indication in which said zinc sulfate, betaine, or a mixture thereof has regulatory approval or institutional review board approval for administration.

An aspect of the present disclosure provides a method of treating COVID-19 in a human patient comprising administering an NAD booster to said patient with less than the minimum effective dose of zinc sulfate, betaine, or a mixture thereof. In some embodiments, the minimum effective dose of zinc sulfate is about 50 mcg/kg, about 40 mcg/kg, or about 30 mcg/kg. In some embodiments, the amount of zinc sulfate administered is less than about 50 mcg/kg per day, less than about 40 mcg/kg, or less than about 30 mcg/kg. In some embodiments, the amount of zinc sulfate administered is less than about 3.5 mg per day, less than about 3 mg per day, or less than about 2 mg per day. In some embodiments, the minimum effective dose of betaine is about 75 mg/kg per day, about 90 mg/kg per day, or about 100 mg/kg per day. In some embodiments, the amount of betaine administered is less than about 75 mg/kg per day, less than about 90 mg/kg per day, or less than about 100 mg/kg per day betaine. In some embodiments, the amount of betaine administered is less than about 5 grams per day, less than about 6 grams per day, or less than about 7 grams per day betaine. In some embodiments, an amount of zinc sulfate greater than zero but less than the minimum effective dose is administered. In some amounts, no zinc sulfate is administered. In some embodiments, an amount of betaine greater than zero but less than the minimum effective dose is administered. In some embodiments, no betaine is administered.

An aspect of the present disclosure provides a method of treating COVID-19 in a human patient comprising administering an NAD booster to said patient, wherein said patient that has not received azithromycin as a treatment for COVID-19 prior to initial administration of NAD booster.

An aspect of the present disclosure provides a method of treating COVID-19 in a human patient comprising administering an NAD booster to said patient, wherein said patient has not received hydroxychloroquine as a treatment for COVID-19 prior to initial administration of NAD booster.

An aspect of the present disclosure provides a method of treating COVID-19 in a human patient, comprising administering an NAD booster other than NMN or NR to said patient.

In some embodiments said NAD booster is administered in crystal form. In some embodiments said NAD booster is administered in amorphous form. In some embodiments said NAD booster is administered in a solid pharmaceutical dosage form. In some embodiments said NAD booster is administered as a tablet. In some embodiments said NAD booster is administered to reduce or alleviate the symptoms of COVID-19-related cytokine storm in a patient exhibiting symptoms of cytokine storm. In some embodiments said NAD booster is administered to reduce or prevent the incidence of COVID-19-related cytokine storm in a patient diagnosed with COVID-19 but not yet exhibiting symptoms of cytokine storm. In some embodiments said NAD booster is administered to reduce or prevent the incidence of, or to reduce or alleviate the symptoms of, COVID-19-related cytokine storm in a patient at risk of developing COVID-19-related cytokine storm. In some embodiments said risk of developing COVID-19-related cytokine storm is evaluated based on clinical presentation of risk factors, or based on a laboratory-confirmed biomarker associated with a risk of developing COVID-19-related cytokine storm. In some embodiments said patient is admitted in a hospital or in-patient clinic. In some embodiments said patient is not receiving assistance in breathing from a ventilator. In some embodiments said NAD booster is administered in a dose of between about 100 mg and about 4 grams per day. In some embodiments said NAD booster is administered in a dose between about 200 mg and about 2 grams per day. In some embodiments said NAD booster is administered in a dose between about 400 mg and about 1 gram per day. In some embodiments said NAD booster is administered in a dose between about 500 mg and about 2 grams per day. In some embodiments, said NAD booster is administered in a daily regimen selected from once daily to four times daily. In some embodiments said NAD booster is administered in a daily dosage regimen selected from once daily, twice daily, and four times daily. In some embodiments said NAD booster is administered for a period up to 5 days inclusive. In some embodiments said NAD booster is administered for a period greater than 5 days. In some embodiments said NAD booster is nicotinamide mononucleotide (NMN). In some embodiments said NAD booster is an analog of nicotinamide mononucleotide (NMN). In some embodiments said NAD booster is a salt of nicotinamide mononucleotide (NMN). In some embodiments said NAD booster is nicotinamide riboside (NR). In some embodiments said NAD booster is an analog of nicotinamide riboside (NR). In some embodiments said NAD booster is a salt of nicotinamide riboside (NR).

An aspect of the present disclosure provides a method of prophylactic treatment of COVID-19 in a human at risk of contracting COVID-19 or in a human with asymptomatic or mild COVID-19, wherein said human has not received a diagnosis of COVID-19 based on a laboratory-confirmed finding of the presence of SARS-CoV-2 virus, said method comprising administering an NAD booster to said patient in a daily dose between about 100 mg and 4 grams.

An aspect of the present disclosure provides a method of treatment for sequelae of SARS-CoV-2 infection, in particular for treatment of acute kidney injury (AKI). In various aspects, a patient presenting to the clinic or hospital is screened for SARS-CoV-2 infection, and if positive, further screened for kidney function. In various aspects, the screening includes a measurement of serum creatinine levels. If acute kidney injury is observed or suspected, the patient is treated with an NAD booster, for example NMN.

An aspect of the present disclosure is a method of treatment of acute kidney injury (AKI), irrespective of the source of the injury to the kidney. In various aspects, a patient presenting to a clinic, hospital, or medical professional is screened for kidney function, for example by measurement of serum creatinine levels. If acute kidney injury is observed or suspected based on kidney function screening, such as by serum creatinine levels above the normal levels, or by clinical history, the patient is treated with an NAD booster, for example NMN. In some embodiments, treatment with an NAD booster such as NMN is more efficacious than placebo in preventing worsening of kidney function, as assessed by longitudinal changes in serum creatinine concentration. In some embodiments, treatment with an NAD booster such as NMN is more efficacious than placebo in improving circulating (KIM-1 and/or NGAL) and urinary (urinary albumin, KIM-1, and/or NGAL) biomarkers of acute kidney injury. In some embodiments, treatment with an NAD booster such as NMN is more efficacious than placebo in improving one or more inflammatory biomarkers (IL6, TNF-alpha, hsCRP, ferritin, a ratio of angiotensin 2 to angiotensin 1, 7 and ACE2). In some embodiments, the NAD booster such as NMN is more efficacious than placebo for patients with AKI in improving biomarkers of endothelial injury and microvascular thrombosis, including vWF, PAI-1, D-dimer, and fibrinogen, including stage 1 AKI. In some embodiments, the NAD booster such as NMN is more efficacious than placebo for patients with AKI in improving oxygen saturation and markers of acute lung injury (IL6, TNF-alpha, RAGE, and/or surfactant-A), including stage 1 AKI. In some embodiments, the NAD booster such as NMN is more efficacious than placebo for patients with AKI in improving circulating biomarkers of myocardial injury, such as high sensitivity troponin I, including stage 1 AKI.

In some embodiments said NAD booster is nicotinamide mononucleotide (NMN). In some embodiments said NAD booster is an analog of nicotinamide mononucleotide (NMN). In some embodiments said NAD booster is a salt of nicotinamide mononucleotide (NMN). In some embodiments said NAD booster is nicotinamide riboside (NR). In some embodiments said NAD booster is an analog of nicotinamide riboside (NR). In some embodiments said NAD booster is a salt of nicotinamide riboside (NR). In some embodiments, said NAD booster is neither NMN nor NR.

### DETAILED DESCRIPTION

Treatment with compounds that boost NAD levels can be effective in treating subjects with infectious diseases—such as COVID-19 and other viral infections—including those experiencing symptoms such as cytokine release syndrome (cytokine storm). Cytokine release syndrome is an acute systemic inflammatory syndrome that can arise from a variety of causes. In particular, cytokine storms have been described in COVID-19 as well as other severe viral syndromes (SARS, MERS). A subset of patients exhibits notably elevated cytokines, and severe patients can also exhibit much higher levels of IL6, CRP, ferritin, D-dimer, and other markers, as well as lymphopenia (reduced count of CD4+ and CD8+ T cells). For example, in one report, a D-dimer level on admission of >2.0 ug/ml identified a subset of patients likely to die (12/67>=2.0 vs 1/267<2.0, sensitivity 92.3%, specificity 83.3%) (D-dimer levels on admission to predict in-hospital mortality in patients with Covid-19. Zhang L, Yan X, Fan Q, et al. J Thromb Haemost. 2020 Apr. 19. doi: 10.1111/jth.14859). NAD modulates the NLRP3 inflammasome release of IL-1β, by which it may modulate the cytokine storm. NAD levels are known to decline with age, which may also contribute to worse outcomes for older COVID-19 patients.

Treatment with compounds that boost NAD levels can be effective in treating specific symptoms or sequelae in subjects with infectious diseases, such as COVID-19 and other viral infections, including sequelae relating to acute kidney injury.

Treatment with compounds that boost NAD levels can be effective in treating specific symptoms or sequelae in subjects with acute kidney injury irrespective of the cause of the acute kidney injury.

In other embodiments, provided herein is a method for treating a cardiovascular disease by administering to a subject in need thereof a disclosed compound and/or a pharmaceutical composition thereof. Cardiovascular diseases that can be treated or prevented include cardiomyopathy or myocarditis, such as alcoholic cardiomyopathy, drug induced cardiomyopathy, ischemic cardiomyopathy, and hypertensive cardiomyopathy; coronary artery disease or myocardial infarction; coronary heart disease; angina pectoris; congestive heart failure; stroke; cognitive functions in dementia; retinopathy; peripheral neuropathy; nephropathy; nephrotic syndrome; hypertensive nephrosclerosis; atheromatous disorders of the major blood vessels (macrovascular disease) such as the aorta, the coronary arteries, the carotid arteries, the cerebrovascular arteries, the renal arteries, the iliac arteries, the femoral arteries, the popliteal arteries, cardiac arterioles, and associated capillary beds of the eye, the kidney, the heart, and the central and peripheral nervous systems; coronary and/or peripheral arterial disease; ischaemia; heart disease; and vascular disease. In various embodiments, provided herein are methods for treating HFpEF (also known as Heart Failure with preserved Ejection Fraction). In various embodiments, a subject is diagnosed with HFpEF prior to administration of NMN or an analog thereof.

### NAD Boosters

A variety of NAD boosters can be useful for methods of the present disclosure. NAD boosters include, but are not limited to, nicotinamide mononucleotide (NMN), nicotinic acid mononucleotide (NaMN), nicotinamide riboside (NR), and salts thereof. In various embodiments, NR is excluded from the group of NAD boosters.

NMN can be amorphous or crystalline. Crystalline forms of NMN are described in U.S. Pat. Nos. 10,392,415, 10,233,208, 10,392,416, each of which is incorporated by reference in its entirety. Crystal forms can be advantageous over amorphous forms, for example due to increased purity and increased stability.

Analogs of other NAD boosters can also be employed. For example, analogs of compounds such as NMN are described in U.S. Pat. Nos. 9,855,289, 9,919,003, 10,548,913, and 10,618,927, each of which is incorporated by reference in its entirety.

### Methods of Treatment and Administration of Compounds

Provided herein are methods for using the disclosed compounds and pharmaceutical compositions thereof.

In some embodiments, the disclosed compounds and pharmaceutical compositions thereof can be useful for a variety of therapeutic applications relating to SARS-CoV-2 infection, its symptoms and sequelae, and acute kidney injury, whether or not related to SARS-CoV-2 infection. Such symptoms and sequelae include, for example, treating and/or reducing a wide variety of diseases and disorders including, for example, diseases or disorders related to aging or stress, diabetes, obesity, neurodegenerative diseases, ataxia and related muscle disorders, acute organ failure, cardiovascular disease, blood clotting disorders, inflammation, cancer, long-lasting complications from prior SARS-CoV-2 infection, and/or flushing, etc. The methods comprise administering to a subject in need thereof a disclosed compound and/or pharmaceutical composition thereof.

NAD boosters, such as those disclosed herein, can be administered to subjects in need thereof. An NAD booster can be administered alone, in combination with other NAD boosters, or in combination with other compounds.

In some cases, NAD boosters are administered to subjects in need thereof in the absence of one or more particular compounds. In an example, an NAD booster is administered in the absence of administration of one or more of zinc sulfate, betaine, azithromycin, chloroquine, or hydroxychloroquine. In another example, an NAD booster is administered in the absence of administration of at least a minimum effective dose or minimum recommended dose of one or more of zinc sulfate, betaine, azithromycin, chloroquine, or hydroxychloroquine. In some embodiments, the minimum effective dose of zinc sulfate is about 50 mcg/kg, about 40 mcg/kg, or about 30 mcg/kg. In some embodiments, the amount of zinc sulfate administered is less than about 50 mcg/kg per day, less than about 40 mcg/kg, or less than about 30 mcg/kg. In some embodiments, the amount of zinc sulfate administered is less than about 3.5 mg per day, less than about 3 mg per day, or less than about 2 mg per day. In some embodiments, the minimum effective dose of betaine is about 75 mg/kg per day, about 90 mg/kg per day, or about 100 mg/kg per day. In some embodiments, the amount of betaine administered is less than about 75 mg/kg per day, less than about 90 mg/kg per day, or less than about 100 mg/kg per day betaine. In some embodiments, the amount of betaine administered is less than about 5 grams per day, less than about 6 grams per day, or less than about 7 grams per day betaine.

In some cases, NAD boosters are administered to a subset of subjects in need thereof, wherein such subjects in the subset are proven or suspected of being infected with SARS-CoV2 and are experiencing or at risk of experiencing acute kidney injury.

In some cases, NAD boosters are administered to a subject in need thereof, wherein said subject is experiencing or at risk of experiencing acute kidney injury, and such treatment stabilizes or improves kidney function according to a laboratory-confirmed measurement, such as serum creatinine levels.

In some cases, disclosed herein are methods of treating sequelae of SARS-CoV-2 infection in a human patient diagnosed with COVID-19, comprising administering an active agent selected from NMN or an analog thereof, wherein said analog is not NR, to said human patient. In some cases, said sequelae are selected from acute lung injury, cardiac pathologies, neurological sequelae, neurodegenerative diseases, ataxia and related muscle disorders, acute organ failure, cardiovascular disease, blood clotting disorders, inflammation, cancer, and/or flushing. In some embodiments, said treatment occurs in whole or in part following a negative test for the presence of active coronavirus. In some embodiments, said treatment occurs in an outpatient setting.

In some cases, provided herein is a method of treatment of acute kidney injury (AKI), irrespective of the source of the injury to the kidney, including stage 1, stage 2, stage 3, stage 4, or stage 5 AKI. In some cases, provided herein are methods for stabilizing or improving renal function. Such methods may include treatment of subjects with stage 1 chronic kidney disease, stage 2 chronic kidney disease, stage 3 chronic kidney disease, stage 4 chronic kidney disease, or stage 5 chronic kidney disease. Such methods may include treatment of subjects with end-stage renal failure.

In some cases, provided herein is a method for treating a cardiovascular disease by administering to a subject in need thereof a disclosed compound and/or a pharmaceutical composition thereof. Cardiovascular diseases that can be treated or prevented include cardiomyopathy or myocarditis, such as alcoholic cardiomyopathy, drug induced cardiomyopathy, ischemic cardiomyopathy, and hypertensive cardiomyopathy; coronary artery disease or myocardial infarction; coronary heart disease; angina pectoris; congestive heart failure; stroke; cognitive functions in dementia; retinopathy; peripheral neuropathy; nephropathy; nephrotic syndrome; hypertensive nephrosclerosis; atheromatous disorders of the major blood vessels (macrovascular disease) such as the aorta, the coronary arteries, the carotid arteries, the cerebrovascular arteries, the renal arteries, the iliac arteries, the femoral arteries, the popliteal arteries, cardiac arterioles, and associated capillary beds of the eye, the kidney, the heart, and the central and peripheral nervous systems; coronary and/or peripheral arterial disease; ischaemia; heart disease; and vascular disease. In various embodiments, the NMN or analog thereof is not nicotinamide riboside (NR).

In some cases, provided herein are methods for treatment of lung function, including chronic lung injury, any type of acute lung injury, pulmonary infiltrates, or lung pathologies.

In some cases, provided herein are methods for treatment of hyperinflammation.

### Compositions

Also provided herein are compositions comprising one or more pharmaceutically acceptable excipients and one or more NAD boosters, such as nicotinamide mononucleotide.

In some embodiments, the composition is in a solid form selected from a tablet, a pill, a capsule, a caplet, a troche, granules, powders, sachet, dry powder inhalation form, a chewable, a pastille, and a lozenge. In certain embodiments, the composition is in the form of a tablet. In other embodiments, the composition is in a form of a hard or soft gelatin capsule.

In some embodiments, the compound is in an amorphous solid form. In other embodiments, the compound is in a crystalline solid form.

In some embodiments, the amount of NAD booster in the composition is about 0.001% by weight to about 95% by weight, about 0.01% by weight to about 50% by weight, about 0.1% by weight to about 30% by weight, about 0.25% by weight to about 10% by weight, or about 0.5% by weight to about 5% by weight.

In some embodiments, the pharmaceutically acceptable excipient is selected from an anti-adherent, binder, coating, dye, disintegrant, flavoring agent, glidant, lubricant, preservative, sorbent, sweetener, syrups, elixirs, dispersant, diluent, filler, granulating agent, coating agent, wax, suspending agent, wetting agent, thickener and vehicle and combinations thereof. In some embodiments, the excipient is a solid excipient.

In some embodiments, the pharmaceutically acceptable excipient is present in an amount of at least about 5% by weight, at least about 10% by weight, at least about 15% by weight, at least about 20% by weight, at least about 25% by weight, at least about 30% by weight, at least about 35% by weight, at least about 40% by weight, at least about 45% by weight, at least about 50% by weight, at least about 55% by weight, or at least about 60% by weight of the composition. In some embodiments, the pharmaceutically acceptable excipient is present in an amount of at least about 20% by weight, at least about 25% by weight, at least about 30% by weight, at least about 35% by weight, or at least about 40% by weight, preferably at least about 30% by weight of the composition. In other embodiments, the pharmaceutically acceptable excipient is present in an amount of at least about 50% by weight of the composition.

In some embodiments, the NAD booster is an active pharmaceutical ingredient selected from compounds in the NAD+ pathway, such as nicotinic acid (NA), nicotinamide (Nam), nicotinamide mononucleotide (NMN), nicotinamide riboside (NR), nicotinic acid mononucleotide (NaMN), nicotinic acid riboside (NAR), nicotinamide adenine dinucleotide (NAD⁺/NADH), nicotinamide adenine dinucleotide phosphate (NADP), and nicotinic acid adenine dinucleotide (NaAD). In some embodiments, the active pharmaceutical ingredient is an amorphous solid. In some embodiments, the active pharmaceutical ingredient is a crystalline solid. In some embodiments, the active pharmaceutical ingredient is amorphous NMN.

The present invention also includes useful forms of the compounds of the present invention, such as metabolites, hydrates, solvates, prodrugs, salts, pharmaceutically acceptable salts, and/or co-precipitates.

The compounds of the present invention can exist as a hydrate, or as a solvate, wherein the compounds of the present invention form a crystal that contains molecules of polar solvents, in particular water, methanol or ethanol, for example, as structural element of the crystal lattice of the compounds. The molecules of polar solvents, in particular water, may be present in a stoichiometric or non-stoichiometric ratio with the molecules of the compound. In the case of stoichiometric solvates, e.g., a hydrate, hemi-, (semi-), mono-, sesqui-, di-, tri-, tetra-, penta-, etc. solvates or hydrates, respectively, are possible. The present invention includes all such hydrates or solvates.

The phrase “pharmaceutically acceptable salt” means a salt that is pharmaceutically acceptable. Examples of pharmaceutically acceptable salts include, but are not limited to: (1) acid addition salts, formed with inorganic acids such as hydrochloric acid, hydrobromic acid, sulfuric acid, nitric acid, phosphoric acid, and the like; or formed with organic acids such as glycolic acid, pyruvic acid, lactic acid, malonic acid, malic acid, maleic acid, fumaric acid, tartaric acid, citric acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, lauryl sulfuric acid, gluconic acid, glutamic acid, salicylic acid, muconic acid, and the like or (2) basic addition salts formed with the conjugate bases of any of the inorganic acids listed above, wherein the conjugate bases comprise a cationic component selected from among Na⁺, Mg²⁺, Ca²⁺, NH_{g}R_{4-g}⁺, in which R is a C₁₋₃ alkyl and g is a number selected from 0, 1, 2, 3, or 4. It should be understood that all references to pharmaceutically acceptable salts include solvent addition forms (solvates) or crystal forms (polymorphs) as defined herein, of the same acid addition salt.

Further, it is possible for the compounds of the present invention to exist in free form, e.g., as a free base, free acid, or zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The present invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts.

In certain embodiments, the compound is a salt with an anion selected from acetate, triflate, halide, trifluoroacetate, or formate. In other embodiments, if the disclosed compound is in contact with a media, e.g., aqueous media, the anion can be selected from, for example, OH⁻, H₂PO₄⁻, HPO₄²⁻, HSO₄⁻, SO₄²⁻, NO₃⁻, HCO₃⁻, and CO₃²⁻.

In some embodiments, the disclosed compounds are in the form of a negatively charged phosphate, which may form a salt with any suitable cation. The cation can alter as the compound is isolated or transferred into media with different anionic species. For example, a disclosed compound may be in the form of a phosphate salt that is a pharmaceutically acceptable salt as described herein. In certain embodiments, the cation can be selected from Li⁺, Na⁺, K⁺, Mg²⁺, and Ca²⁺.

### Pharmaceutical Formulations

The compounds of this invention are formulated with conventional carriers and excipients, which can be selected in accord with ordinary practice. Tablets can contain excipients, glidants, fillers, binders and the like. All formulations will optionally contain excipients such as those set forth in the “Handbook of Pharmaceutical Excipients” (1986). Suitable excipients are also listed in the US Food and Drug Administration Inactive Ingredients Database. Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations can range from about 3 to about 11, but is ordinarily about 7 to about 10.

While it is possible for the active ingredients to be administered alone, it may be preferable to present them as pharmaceutical formulations. The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations of the present invention suitable for oral administration may be presented as discrete units such as capsules, cachets or tablets, each containing a predetermined amount of the active ingredient as a powder or granules. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Pharmaceutical formulations according to the present invention comprise a compound according to the invention together with one or more pharmaceutically acceptable carriers or excipients and optionally other therapeutic agents. Pharmaceutical formulations containing the active ingredient may be in any form suitable for the intended method of administration. When intended for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques, including microencapsulation, to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active material(s) in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia; and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The amount of active ingredient that may be combined with the carrier material to produce a single dosage form will vary depending upon the subject treated and the particular mode of administration. For example, a time-release formulation intended for oral administration to humans may contain approximately 1 to approximately 2000 mg of active material compounded with an appropriate and convenient amount of carrier material which may vary from about 5% to about 95% of the total compositions (weight:weight). The pharmaceutical composition can be prepared to provide easily measurable amounts for administration.

Formulations suitable for intrapulmonary or nasal administration have a particle size for example in the range of about 0.1 to about 500 microns, such as about 0.5, about 1, about 30, or about 35 microns etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

### Definitions

Unless defined otherwise, all technical and scientific terms used herein have the meaning commonly understood by a person skilled in the art of the present disclosure. As used herein, the following terms have the meanings ascribed to them below, unless specified otherwise.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The terms “optional” or “optionally” as used herein means that a subsequently described event or circumstance may but need not occur, and that the description includes instances where the event or circumstance occurs and instances in which it does not. For example, “optional bond” means that the bond may or may not be present, and that the description includes single, double, or triple bonds.

The term “purified,” as described herein, refers to the purity of a given compound. For example, a compound is “purified” when the given compound is a major component of the composition, i.e., at least about 50% w/w pure. Thus, “purified” embraces at least about 50% w/w purity, at least about 60% w/w purity, at least about 70% purity, at least about 80% purity, at least about 85% purity, at least about 90% purity, at least about 92% purity, at least about 94% purity, at least about 96% purity, at least about 97% purity, at least about 98% purity, at least about 99% purity, at least about 99.5% purity, and at least about 99.9% purity, wherein “substantially pure” embraces at least about 97% purity, at least about 98% purity, at least about 99% purity, at least about 99.5% purity, and at least about 99.9% purity.

The term “metabolite,” as described herein, refers to a compound produced in vivo after administration to a subject.

The term “salts,” as described herein, refers to a compound comprising a cation and an anion, which can be produced by the protonation of a proton-accepting moiety and/or deprotonation of a proton-donating moiety. It should be noted that protonation of the proton-accepting moiety results in the formation of a cationic species in which the charge is balanced by the presence of a physiological anion, whereas deprotonation of the proton-donating moiety results in the formation of an anionic species in which the charge is balanced by the presence of a physiological cation.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

The terms “treatment”, “treating”, “palliating” and “ameliorating” are used interchangeably herein. These terms refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made.

The term “preparation” or “dosage form” is intended to include both solid and liquid formulations of the active compound and one skilled in the art will appreciate that an active ingredient can exist in different preparations depending on the desired dose and pharmacokinetic parameters.

The term “excipient” as used herein refers to a compound that is used to prepare a pharmaceutical composition, and is generally safe, non-toxic and neither biologically nor otherwise undesirable, and includes excipients that are acceptable for veterinary use as well as human pharmaceutical use.

The term “minimum effective dose” (MED) as used herein refers to the lowest dose level of a pharmaceutical product that provides a clinically significant response in average efficacy, which is also statistically significantly superior to the response provided by the placebo.

The term “minimum recommended dose” as used herein refers to the lowest dose level of a pharmaceutical product that is recommended for use by a regulatory agency, manufacturer, or medical professional.

The term “institutional review board” (IRB) as used herein refers to a type of committee that applies research ethics by reviewing the methods proposed for research to ensure that they are ethical.

The term “COVID-19” as used herein refers to the disease caused by SARS-CoV-2, including mutational variants.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### Incorporation by Reference

All US patents and US and PCT published patent applications and non-patent literature mentioned in this specification are herein incorporated by reference to the same extent as if each independent patent and publication was specifically and individually indicated to be incorporated by reference.

1. A method of treating a human patient diagnosed with COVID-19 comprising administering an NAD booster to said patient, wherein said NAD booster is administered to said patient according to one or more of the following conditions:
i) in the absence of administration of a dose of zinc sulfate, betaine, or a mixture thereof, equal to or greater than 2 mg of zinc sulfate per day or 5 grams of betaine per day;
ii) in a patient who has not received azithromycin as treatment for COVID-19 prior to initial administration of said NAD booster; or
iii) in a patient who has not received hydroxychloroquine as treatment for COVID-19 prior to initial administration of said NAD booster.

2. A method of treating COVID-19 in a human patient comprising administering an NAD booster to said patient, wherein said NAD booster is administered to said patient in the absence of administration of a dose of zinc sulfate, betaine, or a mixture thereof, equal to or greater than 2 mg of zinc sulfate per day or 5 grams of betaine per day.

3. A method of treating COVID-19 in a human patient comprising administering an NAD booster to said patient, wherein said patient has not received azithromycin as a treatment for COVID-19 prior to initial administration of NAD booster.

4. A method of treating COVID-19 in a human patient comprising administering an NAD booster to said patient, wherein said patient has not received hydroxychloroquine as a treatment for COVID-19 prior to initial administration of NAD booster.

5. The method of any one of claims 1-4, wherein said NAD booster is administered in crystal form.

6. The method of any one of claims 1-4, wherein said NAD booster is administered in amorphous form.

7. The method of any one of the above claims, wherein said NAD booster is administered in a solid pharmaceutical dosage form.

8. The method of claim 7, wherein said NAD booster is administered as a tablet.

9. The method of any one of claims 1-8, wherein said NAD booster is administered to reduce or alleviate the symptoms of COVID-19-related cytokine storm in a patient exhibiting symptoms of cytokine storm.

10. The method of any one of claims 1-8, wherein said NAD booster is administered to reduce or prevent the incidence of COVID-19-related cytokine storm in a patient diagnosed with COVID-19 but not yet exhibiting symptoms of cytokine storm.

11. The method of any one of claims 1-8, wherein said NAD booster is administered to reduce or prevent the incidence of, or to reduce or alleviate the symptoms of, COVID-19-related cytokine storm in a patient at risk of developing COVID-19-related cytokine storm.

12. The method of claim 11, where said risk of developing COVID-19-related cytokine storm is evaluated based on clinical presentation of risk factors, or based on a laboratory-confirmed biomarker associated with a risk of developing COVID-19-related cytokine storm.

13. The method of any one of the above claims, wherein said patient is admitted in a hospital or in-patient clinic.

14. The method of any one of the above claims, wherein said patient is not receiving assistance in breathing from a ventilator.

15. The method of any one of the above claims, wherein said NAD booster is administered in a dose of between about 100 mg and about 4 grams per day.

16. The method of any one of the above claims, wherein said NAD booster is administered in a dose between about 500 mg and about 2 grams per day.

17. The method of any one of the above claims, wherein said NAD booster is administered in a daily dosage regimen selected from once daily, twice daily, and four times daily.

18. The method of any one of claims 1-17, wherein said NAD booster is administered for a period up to 5 days inclusive.

19. The method of any one of claims 1-17, wherein said NAD booster is administered for a period greater than 5 days.

20. The method of any one of the preceding claims, wherein said NAD booster is nicotinamide mononucleotide (NMN).

21. The method of any one of the preceding claims, wherein said NAD booster is an analog of nicotinamide mononucleotide (NMN), wherein the NAD booster is not NMN or NR.

22. The method of any one of the preceding claims, wherein said NAD booster is a salt of nicotinamide mononucleotide (NMN).

23. The method of any one of the preceding claims, wherein said NAD booster is nicotinamide riboside (NR).

24. The method of any one of the preceding claims, wherein said NAD booster is an analog of nicotinamide riboside (NR) wherein the NAD booster is not NR or NMN.

25. The method of any one of the preceding claims, wherein said NAD booster is a salt of nicotinamide riboside (NR).

26. A method of prophylactic treatment of COVID-19 in a human at risk of contracting COVID-19 or in a human with asymptomatic or mild COVID-19, wherein said human has not received a diagnosis of COVID-19 based on a laboratory-confirmed finding of the presence of SARS-CoV-2 virus, said method comprising administering an NAD booster to said patient in a daily dose between about 100 mg and 4 grams.

27. The method of claim 26, wherein said NAD booster is nicotinamide mononucleotide (NMN).

28. The method of claim 26, wherein said NAD booster is an analog of nicotinamide mononucleotide (NMN).

29. The method of claim 26, wherein said NAD booster is a salt of nicotinamide mononucleotide (NMN).

30. The method of claim 26, wherein said NAD booster is nicotinamide riboside (NR).

31. The method of claim 26, wherein said NAD booster is an analog of nicotinamide riboside (NR).

32. The method of claim 26, wherein said NAD booster is a salt of nicotinamide riboside (NR).

33. A method of treating acute kidney injury (AKI) comprising administering an active agent selected from NMN and an analog of NMN, wherein said analog of NMN is not NR, to a subject in need thereof.

34. The method of claim 33, wherein said acute kidney injury is a symptom or result of SARS-CoV-2 infection.

35. The method of claim 34, further comprising testing said subject for SARS-CoV-2 infection and kidney function.

36. The method of claim 33, wherein said acute kidney injury is not a symptom or result of SARS-CoV-2 infection.

37. The method of claim 33, wherein said active agent is nicotinamide mononucleotide (NMN).

38. The method of claim 33, wherein said active agent is an analog of nicotinamide mononucleotide (NMN).

39. The method of claim 33, wherein said active agent is a salt of nicotinamide mononucleotide (NMN).

40. The method of any one of claims 33-39, wherein said active agent is administered in a dose of between about 100 mg and about 4 grams per day.

41. The method of any one of claims 33-39, wherein said active agent is administered in a dose between about 200 mg and about 2 grams per day.

42. The method of any one of claims 33-39, wherein said active agent is administered in a dose between about 400 mg and about 1 gram per day.

43. The method of any one of claims 33-39, wherein said subject is screened for kidney function.

44. The method of claim 43, wherein said screening comprises measuring serum creatinine levels.

45. The method of any one of claims 33-44, wherein treatment with an active agent is more efficacious than placebo in preventing worsening of kidney function, as assessed by longitudinal changes in serum creatinine concentration.

46. The method of any one of claims 33-44, wherein treatment with an active agent is more efficacious than placebo in improving circulating and urinary biomarkers of acute kidney injury.

47. The method of any one of claims 33-44, wherein treatment with an active agent is more efficacious than placebo in improving one or more inflammatory biomarkers.

48. The method of any one of claims 33-44, wherein treatment with an active agent is more efficacious than placebo for patients with AKI in improving biomarkers of endothelial injury and microvascular thrombosis.

49. The method of any one of claims 33-44, wherein treatment with an active agent is more efficacious than placebo for patients with AKI in improving oxygen saturation and markers of acute lung injury.

50. The method of any one of claims 33-44, wherein treatment with an active agent is more efficacious than placebo for patients with AKI in improving circulating biomarkers of myocardial injury.

51. A method of treating sequelae of SARS-CoV-2 infection in a human patient diagnosed with COVID-19, comprising administering an active agent selected from NMN and an analog thereof, wherein said analog is not NR, to said human patient.

52. The method of claim 51, wherein said sequelae is selected from acute lung injury, cardiac pathologies, neurological sequelae, neurodegenerative diseases, ataxia and related muscle disorders, acute organ failure, cardiovascular disease, blood clotting disorders, inflammation, cancer, long-lasting complications from prior SARS-CoV-2 infection, and flushing.

53. The method of claim 51, wherein said treating occurs in whole or in part following a negative test for the presence of active coronavirus.

54. The method of claim 51, wherein said treating occurs in an outpatient setting.
